(19) United States
(12) Patent Application Publication
(10) Pub. No.: US 2012/0079615 A1
(43) Pub. Date: Mar. 29, 2012

(54) KNOCK-IN MICE WITH INDUCIBLE LOSS OF EOSINOPHILS

(76) Inventors: James J. Lee, Scottsdale, AZ (US); Nancy A. Lee, Scottsdale, AZ (US)

(21) Appl. No.: 13/240,128

(22) Filed: Sep. 22, 2011

Related U.S. Application Data
(60) Provisional application No. 61/385,815, filed on Sep. 23, 2010.

(51) Int. Cl. A01K 67/027; C12N 5/10

(52) U.S. Cl. 800/18; 435/354

(57) ABSTRACT

Gene knock-in mice having a targeted insertion of the human diphtheria toxin receptor within the eosinophil peroxidase locus are described. Administering diphtheria toxin to such animals results in the loss of eosinophils in the animal. Accordingly, the knock-in mice and nucleic acid constructs featured in the document can be used to generate eosinophil-deficient transgenic animals that are useful for studying pathologies and treatments relating to tissues and organ systems that typically contain eosinophils.

### CROSS-REFERENCE TO RELATED APPLICATION

This application claims the benefit of U.S. Provisional Application No. 61/385,815, filed Sep. 23, 2010. The content of the foregoing application is hereby incorporated by reference in its entirety.

### TECHNICAL FIELD

This invention relates to transgenic mice, and more particularly to transgenic mice in which the human diphtheria toxin receptor is inserted at the eosinophil peroxidase locus.

### BACKGROUND

The management of asthma has changed significantly over the past decade reflecting the recognition of coincident chronic pulmonary inflammation. The wide variability in etiology and presentation of symptoms is anchored by three common characteristics: reversible variable airflow limitations, specific airway histopathologies, and airway hyperresponsiveness (AHR) (i.e., the development of bronchoconstriction in response to nonspecific inflammatory stimuli) (Bochner et al., Ann. Rev. Immun., 12:295, 1994). The onset and progression of allergic asthma is accompanied by overlapping, and often concurrent, inflammatory responses orchestrated by CD4⁺ T_{H}2 lymphocytes, including T cell mediated help of antigen-specific immunoglobulin production, expression of TH2 proinflammatory cytokines, the release of chemokines, and increases in adhesion molecule receptors on activated vascular endothelial cells. These T cell dependent pulmonary changes are also characterized by cellular infiltrates and the subsequent histopathologies believed to be the underlying cause(s) of the accompanying airway obstruction and lung dysfunction. In particular, the differential recruitment of eosinophils to the airway mucosa and lumen are common features of allergic respiratory disease, occurring in >75% of reported cases (Tomassini et al., J. Allergy Clin. Immunol. 88:365, 1991). This selective recruitment suggests that pulmonary pathologies arise, in part, as a consequence of eosinophil effector functions (EEFs). Indeed, studies have implicated eosinophils as immunoregulative cells modulating the inflammatory response as well as proinflammatory cells whose activities lead to epithelial desquamation, airway smooth muscle perturbation, and tissue remodeling (see for example, Underwood et al., Eur. Resp. Jour., 8:2104, 1995)). The use of mouse models has allowed for the dissection of immune pathways of allergic inflammation, including the definition of causative cell types and the identification of cytokine/chemokine ligands as well as their receptors. Moreover, the ability of these models to develop allergen-induced histopathologies and lung dysfunction has led to the widespread use of mice as models of human allergic inflammation.

### SUMMARY

This document is based on the creation of a line of gene knock-in mice whose eosinophil-less character is an inducible event (i.e., iPHIL mice). The loss of eosinophils in iPHIL mice has no apparent effects on the numbers of other white blood cells and is restricted to specific windows of time. Thus, the inducible eosinophil-less mouse described herein offers an opportunity to test the role of eosinophils in allergen-induced respiratory models of human asthma as well as to permit an understanding of the effectiveness of eosinophil ablation during active disease processes. Furthermore, the ablation of eosinophils as a function of time allows studies of many chronic diseases linked with eosinophils (e.g., parasite infection, gastrointestinal diseases, transplant rejection, and cardiovascular disease associated with eosinophil recruitment and activation).

In one aspect, this document features a knock-in mouse (and tissues and cells of the mouse) that contains an insertion of an exogenous nucleic acid at the eosinophil peroxidase locus, the exogenous nucleic acid encoding a human diphtheria toxin receptor, wherein the expression of the human diphtheria toxin receptor is under control of endogenous eosinophil peroxidase regulatory elements, and wherein upon administration of diphtheria toxin to the mouse for four days, the mouse is substantially free of eosinophils and otherwise retains a normal set of blood cells. The exogenous nucleic acid further can include an internal ribosomal entry site downstream of the nucleotide sequence encoding the human diphtheria toxin receptor. The knock-in mouse can maintain production of endogenous eosinophil peroxidase. The exogenous nucleic acid further can include a nucleic acid encoding a selectable marker, wherein the nucleic acid encoding the selectable marker is located between the nucleic acid encoding the diphtheria toxin receptor and the internal ribosomal entry site. The selectable marker can be selected from the group consisting of puromycin, adenosine deaminase, aminoglycoside phosphotransferase, dihydrofolate reductase, hygromycin-B-phosphotransferase, thymidine kinase, and xanthin-guanine phosphoribosyltransferase. The nucleic acid encoding the selectable marker can be flanked by loxP sites. The knock-in mouse can have a BALB/c background.

In another aspect, this document features progeny of a knock-in mouse, wherein the progeny contain the insertion of an exogenous nucleic acid at the eosinophil peroxidase locus.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 depicts the strategy for making an inducible eosinophil-less strain of mice (iPHIL) by inserting the human Diphtheria Toxin Receptor (DT-R) into the eosinophil peroxidase (EPX) locus. In FIG. 1A, a representation of a partial restriction map of the 12 exons comprising the wild type EPX locus is shown on top. The targeting construct uses homology regions from the EPX locus to insert the human DT-R in-frame with the AUG start codon of the EPX open reading frame. The targeted changes also inserts an internal ribosomal entry site (IRES) permitting translation of the otherwise intact EPX open reading frame from the dicistronic mRNA produced by the iPHIL/Recombinant EPX targeted allele. FIG. 1B depicts the locus after a cross of iPHIL/Recombinant EPX mice derived from the gene knock-in ES cells with a CRE deleter animal produces iPHIL mice.

FIG. 2 depicts that administration of diphtheria toxin (15 ng/gm of body weight (i.p.)) results in the ablation of eosinophils from peripheral blood that is both absolute and reversible. FIG. 2A depicts a time course of eosinophil ablation from circulation of allergen-naive iPHIL mice administered diphtheria toxin on two consecutive days. Eosinophil ablation occurs for 4-5 days before returning to wild type levels. FIG. 2B is a bar graph showing that diphtheria toxin administration of iPHIL mice during a one month ovalbumin (OVA) acute sensitization/challenge protocol leads to the ablation of eosinophils and a correspondingly significant decrease in OVA-induced bronchoalveolar lavage cellularity. * p<0.05

FIG. 3 is a schematic of diphtheria toxin administration strategies to iPHIL using an acute OVA sensitization/aerosol challenge protocol. In Experiment #1, Diphtheria toxin is administered to ablate eosinophils during the sensitization phase of the protocol. In Experiment #2, eosinophil ablation in iPHIL mice is limited to the challenge phase of the acute OVA protocol.

FIG. 4 is a schematic of diphtheria toxin administration strategies to iPHIL using a chronic OVA sensitization/aerosol challenge protocol. In FIG. 4A, diphtheria toxin is administered to iPHIL mice ablating eosinophils during the time window of each repetitive OVA aerosol challenge of the protocol. In FIG. 4B, eosinophil ablation in iPHIL mice is limited to the time intervals between the repetitive OVA challenges when eosinophils accumulate in the lung parenchyma.

### DETAILED DESCRIPTION

This document features transgenic mice containing an exogenous nucleic acid encoding a human diphtheria toxin receptor (DT-R) inserted at the eosinophil peroxidase locus. Such animals are also referred to as “knock-in” animals in this document. The amino acid sequence of the human DT-R can be found in GenBank under Accession No. AAN46738.1 [MKLLPSVVLKLFLAAVLSALVTGESLERLRRGLAAGTSNPDPPTVSTDQLLPLG GGRDRKVRDLQEADLDLLRVTLSSKPQALATPNKEEHGKRKKKGKGLGKKRDP CLRKYKDFCIHGECKYVKELRAPSCICHPGYHGERCHGLSLPVENRLYTYDHTTI LAVVAVVLSSVCLLVIVGLLMFRYHRRGGYDVENEEKVKLGMTNSH, SEQ ID NO:1]. Insertion of the DT-R at the eosinophil peroxidase locus results in the expression of the DT-R only in eosinophil-lineage committed cells. Murine cells, unlike primate cells, are insensitive to killing by diphtheria toxin (DT), a heterodimeric protein that depends on receptor-mediated endocytosis to enter cells. See, e.g., Jung et al., Immunity, 17:211-220 (2002); and Pappenheimer et al., J. Infect. Dis. 145:94-102 (1982). In particular, DT enters cells via interaction of its DT B subunit with the cellular DT-R, also known as the heparin binding EGF-like growth factor (hbEGF). Naglich et al., Cell, 69:1051-1061 (1992). Upon endocytosis, the DT A subunit is released and catalyzes the ribosylation of elongation factor 2, resulting in inhibition of protein synthesis and induction of apoptosis in both mitotic and terminally differentiated cells. Murine DT-R is incapable of binding DT, resulting in the resistance of murine cells to DT. However, primate DT-R (e.g., human DT-R or simian DT-R, GenBank Accession No. M93012) efficiently binds to DT. As such, introducing the nucleic acid encoding human DT-R at a particular locus within the murine genome confers sensitivity to the cells encoding human DT-R. Similarly, introducing the nucleic acid encoding simian DT-R at a particular locus within the murine genome confers sensitivity to the cells encoding simian DT-R. Upon administration of DT to the knock-in mice described herein, eosinophil peroxidase expressing marrow cells (i.e., only eosinophil lineage committed progenitor cells) are targeted and die. Conversely, once DT administration is stopped, these eosinophil-lineage committed cells are no longer targeted, allowing production of terminally differentiated peripheral blood eosinophils to resume. Animals can be heterozygous and have insertion of the DT-R at one eosinophil peroxidase allele, or can be homozygous and have insertions at two eosinophil peroxidase alleles.

This document also features progeny and cells of such knock-in animals, provided that the progeny retain the insertion of the DT-R. For example, a transgenic mouse can be used to breed additional mice that contain the insertion (e.g., mice of a different genetic background, such as BALB/c). Tissues and cells obtained from the transgenic mice also are provided herein. As used herein, “derived from” indicates that the cells can be isolated directly from the transgenic mouse or can be progeny of such cells. For example, brain, lung, liver, pancreas, heart, muscle, kidney, thyroid, corneal, skin, blood vessels or other connective tissue can be obtained from a transgenic mouse described herein. Blood and hematopoietic cells, heart cells, hepatocytes, kidney cells, and cells from other organs and body fluids, for example, also can be derived from mice described herein.

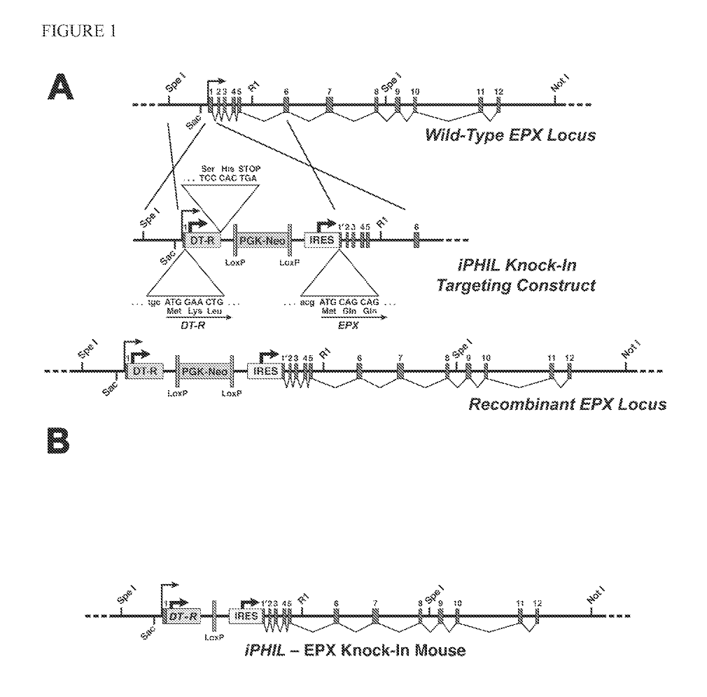

Constructs for inserting an exogenous nucleic acid encoding a DT-R (e.g., the human DT-R) by homologous recombination can be made by targeting the eosinophil peroxidase gene. See, Shastry, Mol. Cell Biochem., 181(1-2):163-179, 1998, for a review of gene targeting technology. For example, as shown in FIG. 1A, a targeting construct can include the nucleic acid encoding the DT-R inserted at the initial codon of the eosinophil peroxidase open reading frame. In addition, the targeting construct includes sequences homologous to the sequences flanking the desired insertion site. It is not necessary for the flanking sequences to be immediately adjacent to the desired insertion site.

An exogenous nucleic acid for targeting the eosinophil peroxidase gene also can include a nucleic acid sequence encoding a selectable marker. Suitable markers for positive drug selection include, for example, the aminoglycoside 3′ phosphotransferase gene (e.g., PGK-promoter-Neo^{R} cassette) that imparts resistance to geneticin (G418, an aminoglycoside antibiotic), and other antibiotic resistance markers, such as the hygromycin-B-phosphotransferase gene that imparts hygromycin resistance. Other selection systems include negative-selection markers such as the thymidine kinase (TK) gene from herpes simplex. Constructs utilizing both positive and negative drug selection also can be used. For example, a construct can contain the aminoglycoside phosphotransferase gene and the TK gene. In this system, cells are selected that are resistant to G418 and sensitive to gancyclovir. Any selectable marker suitable for inclusion in a targeting construct is within the scope of the present invention.

In some embodiments, a sequence encoding a selectable marker can be flanked by recognition sequences for a recombinase such as, e.g., a Cre or Flp recombinase. For example, the selectable marker can be flanked by loxP recognition sites (34 bp recognition sites recognized by the Cre recombinase) or FRT recognition sites such that the selectable marker can be excised from the construct. See, Orban, et al., Proc. Natl. Acad. Sci. (1992) 89 (15): 6861-6865, for a review of Cre/lox technology, and Brand and Dymecki, Dev. Cell (2004) 6(1):7-28. In one embodiment, the selectable marker is excised from the eosinophil peroxidase locus by crossing the mouse containing the DT-R insertion to a mouse expressing the desired recombinase (e.g., Cre or Flp). Such mice are commercially available from, for example, The Jackson Laboratory (Bar Harbor, Me.).

Additional elements that may be useful in nucleic acid constructs, include, but are not limited to, polyadenylation sequences, translation control sequences (e.g., an internal ribosome entry segment, IRES), enhancers, inducible elements, or introns. For example, an IRES element can be used in a targeting construct described herein such that expression of eosinophil peroxidase is maintained. Such elements may not be necessary, although they may increase expression by affecting transcription, stability of the mRNA, translational efficiency, or the like. Additional elements can be included in a nucleic acid construct as desired to obtain optimal expression of the nucleic acids in the cell(s). Sufficient expression, however, can sometimes be obtained without such additional elements.

Cells capable of giving rise to at least several differentiated cell types are “pluripotent”. Pluripotent cells capable of giving rise to all cell types of an embryo, including germ cells, are hereinafter termed “totipotent” cells. Totipotent murine cell lines (embryonic stem or “ES” cells) have been isolated by culture of cells derived from very young embryos (blastocysts). Such cells are capable, upon incorporation into an embryo, of differentiating into all cell types, including germ cells, and can be employed to generate mice described herein. That is, cultured ES cells can be transformed with a targeting construct and cells selected in which the human DT-R is inserted at the eosionophil peroxidase locus.

Nucleic acid constructs can be introduced into ES cells, for example, by electroporation or other standard technique. Selected cells can be screened for gene targeting events. For example, the polymerase chain reaction (PCR) can be used to confirm the presence of the transgene. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA (reverse-transcriptase PCR, RT-PCR), including sequences from total genomic DNA or total cellular RNA. Primers are typically 14 to 40 nucleotides in length, but can range from 10 nucleotides to hundreds of nucleotides in length. PCR is described, for example in PCR Primer: A Laboratory Manual, Ed. by Dieffenbach, C. and Dveksler, G., Cold Spring Harbor Laboratory Press, 1995. At the blastocyst stage, embryos can be individually processed for analysis by PCR, Southern hybridization and splinkerette PCR (see, e.g., Dupuy et al., Proc Natl Acad Sci USA (2002) 99(7):4495-4499).

The ES cells further can be characterized to determine the number of targeting events. For example, genomic DNA can be harvested from ES cells and used for Southern analysis. See, for example, Section 9.37-9.52 of Sambrook et al., “Molecular Cloning, A Laboratory Manual, second edition, Cold Spring Harbor Press, Plainview; NY, 1989.

To generate a knock-in animal, ES cells having at least one insertion are incorporated into a developing embryo. This can be accomplished through injection into the blastocyst cavity of a murine blastocyst-stage embryo, by injection into a morula-stage embryo, by co-culture of ES cells with a morula-stage embryo, or through fusion of the ES cell with an enucleated zygote. The resulting embryo is raised to sexual maturity and bred in order to obtain animals, whose cells (including germ cells) carry the insertion. If the original ES cell was heterozygous for the insertion, several of these animals can be bred with each other in order to generate animals homozygous for the insertion.

Expression of a nucleic acid sequence encoding DT-R in the transgenic mice can be assessed using techniques that include, without limitation, Northern blot analysis of tissue samples obtained from the animal, in situ hybridization analysis, Western analysis, immunoassays such as enzyme-linked immunosorbent assays, and reverse-transcriptase PCR (RT-PCR). Total blood cell counts or the composition of the white cells before or after administration of DT can be assessed using, for example, peripheral blood or bone marrow samples. As described herein, DT administration to a knock-in mouse described herein selectively ablates cells of the eosinophil lineage while maintaining normal ranges of other blood cells. For example, upon administering DT, a mouse described herein has no histologically detectable eosinophils while maintaining a substantially normal level of other blood cells including red blood cells (RBCs). A “substantially normal level of RBCs” is the number of RBCs in a healthy mouse (e.g., about 10-13 RBC/mm³ of blood (×10⁶).

### Methods of Using Knock-In Mice

Antigen-induced mouse models of pulmonary allergic disease have proven particularly informative in the dissection of inflammatory pathways in the lung. Typically, these models involve sensitization with a specific antigen (e.g., ovalbumin OVA) followed by airborne administration of the same antigen. Blyth et al., Am. J. Respir. Cell Mol. Biol., 14(5):425-438 (1996). Sensitized mice treated with aerosolized allergen develop leukocytic infiltrates of the airway lumen dominated by CD4+ lymphocytes and eosinophils. These mice also develop many of the changes pathognomonic of asthma including AHR and goblet cell hyperplasia with excessive mucus production.

In general, OVA-induced type I hypersensitivity in the lung shares many characteristics with human atopic asthma, although specific issues influence conclusions drawn from these models: (i) Immunologic differences exist between mice and humans. For example, while antigen-mediated airway responses in the mouse can be mediated by IgG₁, it appears likely that human responses are restricted to IgE pathways. This difference is reflected in observations that mouse eosinophils lack FcγRII and FcγRI, the low and high-affinity IgE receptors, respectively; (ii) Results from OVA mouse models vary based on the protocol used and the genetic strain of the experimental animals. Choices for both parameters are often based on predetermined experimental endpoints, e.g., some strains of mice display significantly greater levels of AHR and some protocols maximize and/or minimize the roles of IgE and mast cells.

Despite these differences, OVA models have been productively used to examine the details of the underlying molecular and cellular events associated with pulmonary inflammation. These studies have demonstrated that inflammatory pathologies of the lung are dependent on both T cell dependent eosinophil-mediated effector functions and immunoglobulin/mast cell dependent pathways. The relative importance of each pathway, as well as the interactions between them, are not fully understood. Moreover, eosinophil-mediated effector functions still remain a vague description of activities that are apparently critical to the onset/progression of pulmonary pathology.

Administering DT to knock-in mice described herein permits the eosinophil lineage to be inducibly ablated within the animal. Thus, otherwise normal mice substantially free of the eosinophil lineage can be inducibly generated, allowing the role(s) of the eosinophil in the onset/progression of allergic pulmonary pathology and other to be addressed.

Knock-in mice that are substantially free of eosinophils can be sensitized with OVA and then challenged with OVA to induce histopathological changes. For example, alum-precipitated OVA can be injected intraperitoneally on day 0 and on day 5. On day 11, the transgenic mice can be challenged for one hour by exposure to 0.5% OVA by nebulization. OVA-induced histopathologic pulmonary changes can be assessed using standard histological and pulmonary function techniques and compared with non-transgenic OVA sensitized/challenged mice. For example, general lung morphology can be assessed with hematoxylin and eosin staining Goblet cell hyperplasia and mucus production can be assessed with alcian blue (pH 5.2) staining Extracellular matrix protein deposition, for example, deposition of collagen, can be assessed by Masson's trichrome staining.

Changes in the lung immune microenvironment, such as an inflammatory response, T_{H}1 and T_{H}2 responses, eosinophil-induced neutrophil recruitment, and modulation of T_{H}1/T_{H}2 lymphocyte subtypes can be determined by measuring cytokine levels in bronchial alveolar lavage (BAL) fluid. For example, levels of tumor necrosis factor-α, interleukin-1, -2, -4, -5, -8, -10 and 12 can be determined. FACS analysis can be used to assess specific subpopulations of leukocytes recruited to the interstitium and airway lumen. B-cell function and eosinophil-responsive humoral changes can be assessed by measurements of immunoglobulin (Ig) isotypes such as IgG1, IgA, and IgE.

The knock-in mice described herein also can be used to investigate the physiology of any tissue or organ where eosinophils are located (e.g., lung, uterus, intestines, and thymus). For example, the mice can be useful for the investigation of uterine disorders such as disorders resulting in infertility or low fertility. Eosinophil-deficient mice also can be useful for the investigation of gut disorders, including disorders of the intestines, or for autoimmune disorders associated with the thymus. For example, to investigate the role of eosinophils in a pulmonary physiology, a knock-in mouse described herein can be exposed to a pulmonary effector (e.g., an allergen) then lung tissue from the exposed mouse can be compared to lung tissue from a control mouse (e.g., a mouse not exposed to the pulmonary effector or a knock-in mouse described herein not exposed to the pulmonary effector) to identify a role, or a potential role, of eosinophils in pulmonary physiology based, at least in part, on the comparison. Similar methods can be used to investigate the role of eosinophils in other tissues or organs by exposing a knock-in mouse described herein to a test compound and comparing the tissue of interest from the exposed mouse to that of the control mouse to identify a role, or a potential role, of eosinophils in the tissue or organ of interest based, at least in part, on the comparison.

In addition, the animals described herein can be used to classify a test compound as a positive or negative drug candidate. For example, a knock-in mouse described herein can be administered a test compound and an organ or tissue of the mouse can be examined for a presence, absence, or degree of physiological change. The test compound can be classified as a positive or negative drug candidate based on the presence, absence, or degree of the physiological change.

The invention will be further described in the following examples, which do not limit the scope of the invention described in the claims.

### EXAMPLES

### Example 1

Objective: Development of a mouse model of “on demand” ablation of eosinophils to test hypotheses linking eosinophil activities with the regulation of either pulmonary immune response or remodeling/repair events in the lung following allergen provocation.

Results: An inducible mouse model of eosinophil ablation was developed using eosinophil peroxidase (EPX) genomic clones as part of a gene knock-in approach (FIG. 1A) to provide a specific and defined expression pattern that would be limited to eosinophil-lineage committed cells. Specifically, a targeting construct to elicit homologous recombination in C57BL/6J-derived embryonic stem (ES) cells was developed to mediate the insertion of the open-reading frame encoding the human diphtheria toxin receptor (i.e., the heparin binding epithelial growth factor (Jung et al., Immunity, 17:211-20 (2002)) directly at the AUG start codon of the EPX open reading frame. The value of this insertion lies on the fact that unlike the mouse orthologue which is incapable of binding diphtheria toxin (reviewed in Saito et al., Nat. Biotechnol., 19:746-50 (2001)), the human receptor (DT-R) efficiently binds this cytocidal protein leading to the death of receptor expressing cells in the presence of even small amounts of diphtheria toxin (Collier, Toxicon 39:1793-803 (2001); Yamaizumi, et al., Cell, 15:245-50 (1978); and van Rijt et al., J Exp Med 201:981-91 (2005)). Upon administration of diphtheria toxin to knock-in mice, all eosinophil peroxidase expressing marrow cells (i.e., only eosinophil lineage committed progenitor cells) will be targeted and die. Conversely, once diphtheria toxin administration is stopped, these eosinophil-lineage committed cells will no longer be targeted, leading to the production of terminally differentiated peripheral blood eosinophils. As shown in FIG. 1(A), this knock-in strategy also was configured to maintain EPX expression through the inclusion of an internal ribosomal entry site (IRES) to drive the translation of EPX from the dicistronic mRNA produced at this recombinant EPX locus. That is, the strategy was used to generate an inducible eosinophil-less mouse that was not also an EPX knockout model. The knock-in mice generated with this strategy were subsequently crossed with a “CRE-deleter” animal (The Jackson Laboratory) to eliminate the drug selection cassette (i.e., PGK promoter-Neo^{R}) needed for homologous recombination in the targeted knock-in ES cells. This produced an inducible eosinophil-less mouse (iPHIL). See FIG. 1B.

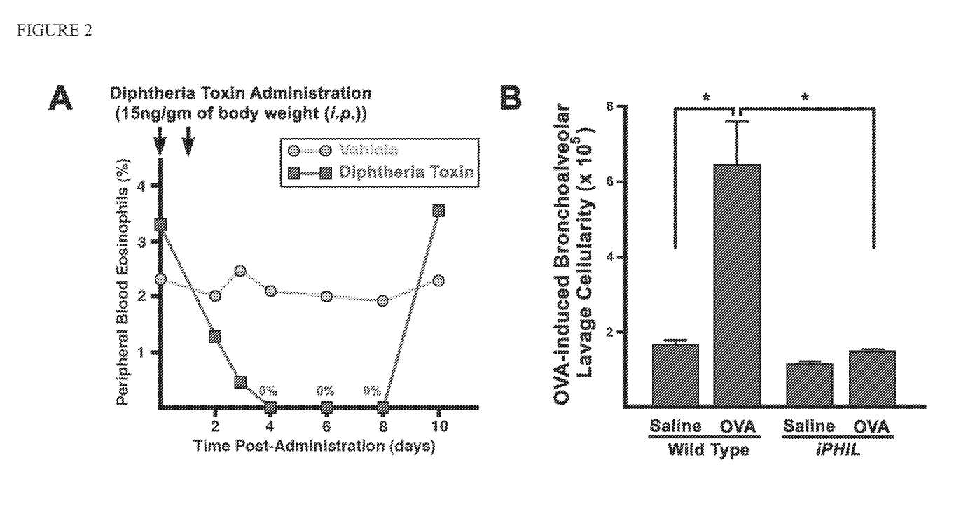

The success and utility of this strategy is demonstrated in the diphtheria toxin administration study presented in FIG. 2A. Two interperitoneal (i.p.) injections of diphtheria toxin (15 ng/gm of body weight) were given on consecutive days to a cohort of mice (n=14) and the presence of eosinophils were determined in the blood and bone marrow as a function of time. These data dramatically showed that within 4 days of the first diphtheria toxin injection, circulating eosinophils levels were reduced to absolute zero and remained at this level for 4-5 days before returning to pre-administration levels. This time course has been repeated and is invariant and reproducible (control animals were either iPHIL mice that received vehicle injections or wild type mice administered diphtheria toxin). Equally important, cell differential analyses of the blood and bone marrow of diphtheria toxin treated mice following ablation of eosinophils showed that only the eosinophil lineage was impacted with no changes occurring in either total blood cell counts or the composition of the remaining white cells. Significantly, ablating eosinophils during a one month long acute ovalbumin (OVA) model demonstrates the ability to ablate eosinophils from the lungs of allergen sensitized/challenged mice (FIG. 2B).

This gene knock-in line of mice provides an essentially wild type mouse that can inducibly become eosinophil-less (e.g., to assess the specific role of eosinophils in immune responses and remodeling events associated with either acute or chronic allergen provocation). This is an advantage over mice that are eosinophil-less during all phases of the allergen sensitization/aerosol challenge protocol, which prevents the identification of specific mechanisms of actions linked to the induced pulmonary allergic responses.

### Example 2

### OVA Sensitization/Aerosol Challenge Protocol

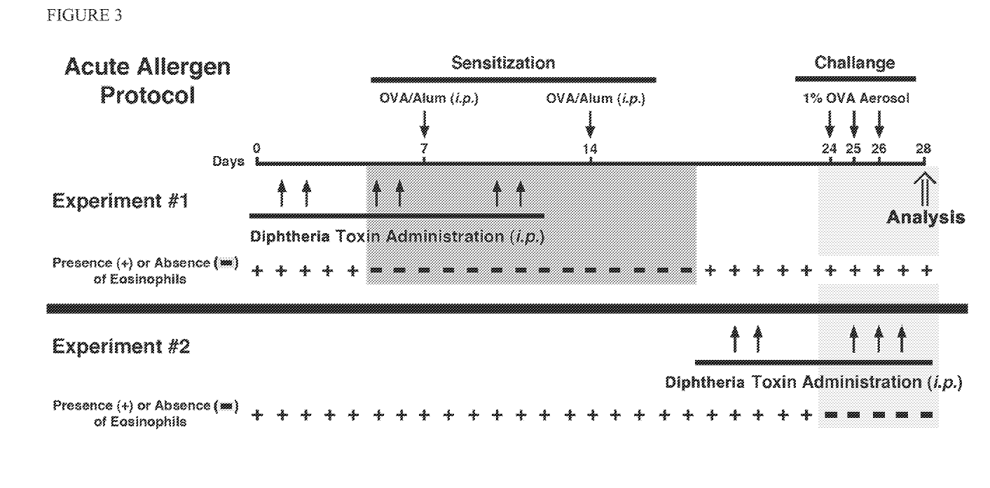

The targeted ablation of eosinophils as a function of time during acute allergic respiratory inflammation is assessed using an OVA sensitization/aerosol challenge model described by Borchers et al., J Immunol 168:3543-3549 (2002), the details of which are presented in FIG. 3. Briefly, mice (20-30 grams) are sensitized by an intraperitoneal injection (1000 of 20 μg chicken ovalbumin emulsified in 2 mg Imject® Alum on days 0 and 14. Mice are subsequently challenged with an aerosol of 1% OVA in saline (OVA) or saline alone (SAL) on days 24, 25, and 26 with the assessment of endpoints of pathology and lung function occurring on day 28 of this protocol. In Experiment #1, diphtheria toxin (15 ng/gm of body weight) is administered (i.p.) to elicit the ablation of eosinophils only during the sensitization phase of the protocol. Inversely, diphtheria toxin will be administered (i.p.) in Experiment #2 to elicit the ablation of eosinophils only during the aerosol challenge phase of the protocol. Leukocyte accumulation in the airways and lung tissue in these studies is determined by bronchoalveolar lavage (BAL) and immunohistochemistry of lung tissue samples, respectively. See, e.g., Borchers et al., 2002, supra; and Borchers et al., Am J Physiol Lung Cell Mol Physiol 285:L114-20 (2003). Cell-free BAL will be flash-frozen in liquid nitrogen and stored at −80° C. for subsequent cytokine level determination by ELISA. Assessments of leukocytes is performed on both peripheral blood and femoral bone marrow as described by Lee et al., J Immunol., 158:1332-1344 (1997). In addition to the kinetics of pro-inflammatory cell recruitment to the lung, other OVA-induced pathologies, such as those summarized Table 1, can be assessed. This table lists specific endpoints to be measured and the approximate number of animals required to determine each parameter.

TABLE 1
Characterization of Ovalbumin (OVA)-induced Pulmonary Pathologies

| | Parameter Assessed | Experimental Rationale | Animals |
|---|---|---|---|
| Induced Histopathologic Pulmonary | Hemotoxylin and Eosin (H&E)* | Preliminary assessment of leukocyte | (5)^{&} |
| Changes | | infiltrates | |
| | Periodic Acid-Schiff* | Goblet cell hyperplasia; mucus | (10)^{&} |
| | | production | |
| | Collagen deposition in lung tissue | Pulmonary fibrosis and the | (10)^{&} |
| | using both immunohistochemistry | deposition of extracellular matrix | |
| | and Picrosirius polarization | (ECM) accompanying induced lung | |
| | | remodeling | |
| | Interstitial lung myofibroblasts as | Assessment of pulmonary | (10)^{&} |
| | double positive-staining cells using | remodeling and fibrosis induced as a | |
| | α-smooth muscle actin (α-SMA) | function of the number of tissue | |
| | and the early smooth muscle | myofibroblasts | |
| | differentiation marker SM-22 | | |
| | IHC using a mAb reactive to α- | Airway smooth muscle hyperplasia | (10)^{&} |
| | SMA | | |
| | IHC using a proliferating cell | Epithelial cell proliferation and ASM | (10)^{&} |
| | nuclear antigen antibody (PCNA) | hyperplasia | |
| | Immunofluorescence of tissue | Identification of eosinophils and | (5)^{&} |
| | sections using an anti-mouse major | degranulation associated with lung | |
| | basic protein polyclonal sera | tissue | |
| Changes in Immune Responses and the Lung | Serum levels of OVA-specific | Assessment of primary immune | (5-10)^{#} |
| Immune Microenvironment | IgG₁ and IgE | responses | |
| | BAL cytokine levels: IFN-γ, IL-4, | Definition of specific pulmonary | (5-10)^{#} |
| | IL-5, IL-13, IL-17, IL-25 | immune responses - Th1: INF-γ; | |
| | | Th17: IL-17; Th2: IL-4, IL-5, -13, - | |
| | | 25 | |
| | BAL levels of unique cyokines and | Growth factors (TGF-β1, FGF, IGF) | (5-10)^{#} |
| | growth factors associated with | or cytokines (IL-1 and IL-6) with | |
| | pulmonary remodeling: IL-1, IL-6, | demonstrated pro-fibrotic activities | |
| | TGF-β1, FGF, IGF | that also were shown by mRNA | |
| | | microarray analyses | |
| Induced Recruitment of Leukocytes | Airway cellular infiltrates | Eosinophil-dependent recruitment of | (5)^{#} |
| To the Lung | (including differentials) derived | leukocytes from the interstitium to | |
| | from BAL fluid | the airway lumen | |
| | FACS analysis of leukocytes | Identification of specific leukocytes | (5-10)^{#} |
| | recruited to lung tissue/airway: | populations recruited to lung tissue/ | |
| | (CD3¹, CD4¹, | airway; (Specificity of Abs: (¹) T | |
| | CD8¹, αβTCR¹, γδTCR¹, B220², | cell; (²) B cell; (³) Macrophage, | |
| | CD19², F4/80³, Mac I³, CCR3⁴, | Eosinophil; (⁴) Eosinophil, (⁵) | |
| | CD11c⁵) | Dendritic cells) | |
| Pathophysiologic Pulmonary Changes | Pulmonary Function: Baseline | Assessments of airway remodeling | (10-20)\hat{ } |
| | Measures of Airway resistance and | and effects on bronchomotor tone | |
| | lung compliance | | |
| | Pulmonary Function: AHR to | Measure of a pathophysiologic | (10-20)\hat{ } |
| | methacholine challenge | response to assess pulmonary | |
| | | inflammation | |

*Specific histopathologic features are scored on a scale of one to five (1 = C57BL/6J mice; 5 = pathologies typical of wild type mice. Scoring is blinded and inter/intra observer reported.
^{&,$$,#,}\hat{ }Represent unique cohorts of animals needed to achieve the objectives indicated in the table

In Experiment #1, eosinophil ablation is limited to the sensitization phase of the acute allergen protocol and thus the eosinophils will be absent during the time when primary immune responses arise. More importantly, eosinophils will be present during the allergen challenge phase of the protocol, eliminating these potential secondary immune responses as a confounding issue. In contrast, eosinophil ablation in Experiment #2 is restricted to the airway challenge phase of the acute allergen protocol. Thus, similar to Experiment #1, eosinophils will remain a normal developmental component of the immunological milieu of these mice before the start of the protocol. However, in Experiment #2, eosinophils will be present throughout the development of primary immune responses (i.e., sensitization phase of the protocol) but uniquely absent during the time window when secondary immune responses and the Th2 polarization of the lung parenchyma develop.

### Example 3

### Chronic Exposure Protocol

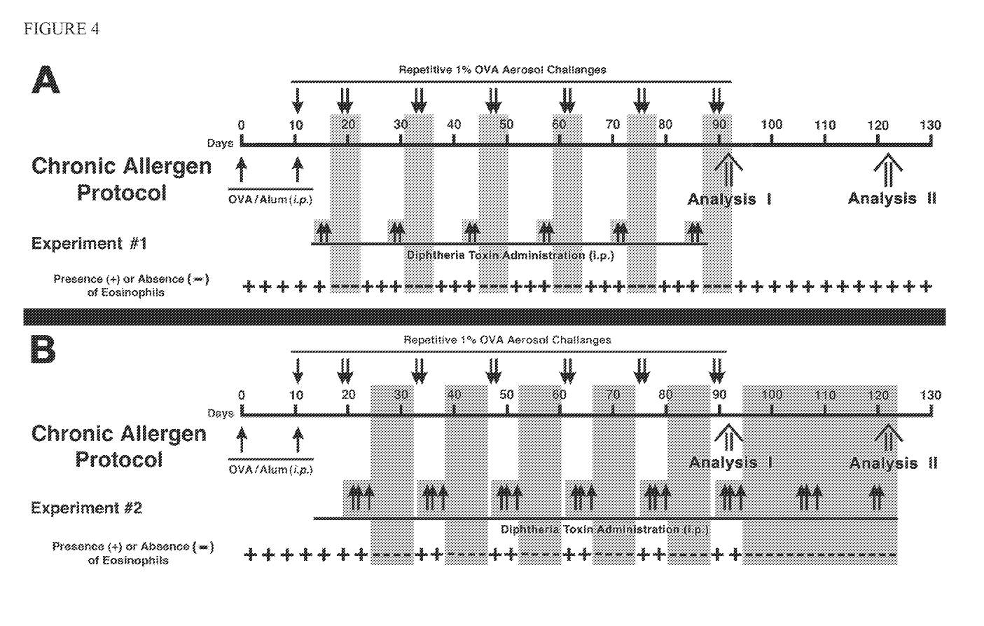

Chronic exposure studies are performed using iPHIL mice that have been backcrossed onto the BALB/c background as the immune responses (and/or induced pathologies) associated with the genetic background BALB/cJ are significantly higher than in the C57BL/6J background. The chronic exposure protocol that is used is represented by the time-line shown in FIG. 4. The strengths of this protocol are the sustained changes induced in both lung structure and function, two key features of human disease. Assessments of induced pathologies is performed at the beginning and end of a one month recovery period, assessing both the induced acute inflammatory responses immediately following allergen provocation as well as the sustained pulmonary responses occurring later.

The design of these studies is intended to delineate between eosinophil activities linked directly with repetitive allergen challenges vs. effector functions derived from the steady-state population of eosinophils accumulating in the lungs as a consequence of repetitive allergen challenge. Thus, in Experiment #1, diphtheria toxin will be administered to iPHIL mice (control animals will include both iPHIL mice receiving vehicle and wild type animals that are administered diphtheria toxin) to ablate eosinophils exclusively within the time windows associated with the chronic allergen challenges See, FIG. 4A. In contrast, diphtheria toxin administration in Experiment #2 inversely promote the ablation of eosinophils only during the intervals between allergen provocation (FIG. 4B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A knock-in mouse comprising an insertion of an exogenous nucleic acid at the eosinophil peroxidase locus, said exogenous nucleic acid encoding a human diphtheria toxin receptor, wherein the expression of said human diphtheria toxin receptor is under control of endogenous eosinophil peroxidase regulatory elements, and wherein upon administration of diphtheria toxin to said mouse for four days, said mouse is substantially free of eosinophils and otherwise retains a normal set of blood cells.

2. The knock-in mouse of claim 1, said exogenous nucleic acid further comprising an internal ribosomal entry site downstream of said nucleotide sequence encoding said human diphtheria toxin receptor.

3. The knock-in mouse of claim 1, wherein said mouse maintains production of endogenous eosinophil peroxidase.

4. The knock-in mouse of claim 2, wherein said exogenous nucleic acid further comprises a nucleic acid encoding a selectable marker, wherein said nucleic acid encoding said selectable marker is located between said nucleic acid encoding said diphtheria toxin receptor and said internal ribosomal entry site.

5. The knock-in mouse of claim 4, wherein said selectable marker is selected from the group consisting of puromycin, adenosine deaminase, aminoglycoside phosphotransferase, dihydrofolate reductase, hygromycin-B-phosphotransferase, thymidine kinase, and xanthin-guanine phosphoribosyltransferase.

6. The knock-in mouse of claim 4, wherein said nucleic acid encoding said selectable marker is flanked by loxP sites.

7. The knock-in mouse of claim 1, wherein said mouse has a BALB/c background.

8. Tissue of said knock-in mouse of claim 1.

9. Cells of said knock-in mouse of claim 1.

10. Progeny of the knock-in mouse of claim 1, wherein said progeny comprises said insertion.


SEQUENCE LISTING (1 sequence)

SEQ ID NO: 1 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH